(19) United States
(12) Patent Application Publication
(10) Pub. No.: US 2011/0033474 A1
(43) Pub. Date: Feb. 10, 2011

(54) INHIBITION OF PLATELET AGGREGATION

(76) Inventors: Desmond Joseph FITZGERALD, Dublin (IE); R. Stephen Porter, Brentwood, TN (US)

Correspondence Address: CLARK & ELBING LLP, 101 FEDERAL STREET, BOSTON, MA, 02110, US

(21) Appl. No.: 12/721,285

(22) Filed: Mar. 10, 2010

Related U.S. Application Data
(63) Continuation of application No. 10/512,189, filed on May 17, 2006, now abandoned.
(60) Provisional application No. 60/374,860, filed on Apr. 23, 2002.

(51) Int. Cl. A61K 39/395; A61K 31/24; A61K 31/4365; A61K 31/225; A61K 31/727; A61K 31/7088; A61P 7/02

(52) U.S. Cl. 424/141.1; 514/534; 514/301; 514/548; 514/56; 514/44 A

(57) ABSTRACT

The present invention provides methods and compositions for preventing platelet aggregation and for treating individuals suffering from conditions or undergoing procedures that may result in unwanted platelet aggregation. In particular the invention provides to methods and compositions for arterial vessel pacification. The methods are based on the administration of a therapeutically effective amount of a glycoprotein IIb/IIIa receptor antagonist, e.g., xemilofiban. The treatment may commence prior to a medical or surgical procedure or an outbreak of a condition, either of which results in the activation of platelets that may lead to thrombus formation, and may continue thereafter.

### CROSS-REFERENCE TO RELATED APPLICATIONS

This application is a continuation of U.S. patent application Ser. No. 10/512,189, filed May 17, 2006, which is the U.S. National Phase of PCT/US03/12515, filed Apr. 23, 2003, which claims benefit of U.S. Provisional Application No. 60/374,860, filed Apr. 23, 2002, the disclosures of which are incorporated by reference in their entirety.

### BACKGROUND OF THE INVENTION

The invention relates to the field of medical treatments and to the use of compounds to produce pharmaceutical agents for use in such medical treatments, in particular the prevention of thrombus formation.

Fibrinogen is a glycoprotein present as a normal component of blood plasma. It participates in platelet aggregation and fibrin formation in the blood clotting mechanism. Platelets are cellular elements found in whole blood, which also participate in blood coagulation. Fibrinogen binding to platelets is important for normal platelet function in the blood coagulation mechanism. When a blood vessel receives an injury, the platelets binding to fibrinogen will initiate aggregation and form a thrombus. Injury can occur during medical or surgical procedures. In addition, certain medical conditions, such as sickle cell anemia crisis, leads to platelet aggregation and thrombosis in vital organs. Interaction of fibrinogen with platelets occurs through a membrane glycoprotein complex, known as glycoprotein IIb/IIIa (GPIIb/IIIa). Inhibitors of this interaction are useful in modulating or preventing platelet thrombus formation.

The activation of platelets and the resultant aggregation have been shown to be important factors in the pathogenesis of acute coronary syndrome, unstable angina pectoris, transient myocardial ischemia, acute myocardial infarction, peripheral arterial occlusion and atherosclerosis. In most of these serious cardiovascular disorders, intracoronary or intra-arterial thrombus is present. The thrombus is generally formed by activated platelets that adhere and aggregate at the site of endothelial injury. Because of the relative contribution of activated platelets to aggregation and subsequent formation of an occlusive thrombus, antiplatelet agents have been developed that inhibit platelet aggregation.

### SUMMARY OF THE INVENTION

The present invention provides methods and compositions for preventing platelet aggregation and for treating individuals suffering from conditions or undergoing procedures that may result in unwanted platelet aggregation. In particular the invention provides methods and compositions for arterial vessel pacification. The methods are based on the administration of a therapeutically effective amount of a glycoprotein IIb/IIIa receptor antagonist, e.g., xemilofiban. The treatment may commence prior to a medical or surgical procedure or an outbreak of a condition, either of which results in the activation of platelets that may lead to thrombus formation, and may continue thereafter.

Accordingly, in one aspect, the invention features a method for inhibiting platelet aggregation in a patient undergoing a medical or surgical procedure. These procedures include, e.g., cardiac interventional procedures, e.g., percutaneous transluminal coronary angioplasty (PTCA), coronary artery stent procedure, or cardiac bypass surgery (CABG), and angioplastic procedures, e.g., atherectomy, balloon angioplasty, laser angioplasty, intracranial angioplasty, and angioplasty of peripheral arteries. Stents (drug coated or non coated) may be inserted as a part of an angioplastic procedure.

The invention features administering to the patient, at least 60 minutes prior to the procedure, a therapeutically effective amount of a glycoprotein IIb/IIIa receptor antagonist, e.g., xemilofiban. Exemplary times for administration include at least 24 hours, and at least 48 hours prior to the cardiac interventional procedure. The administration may also occur no earlier than 72, 48, or 24 hours prior to the procedure. In certain embodiments, the method further includes administering a therapeutically effective amount of a glycoprotein IIb/IIIa antagonist after the cardiac intervention procedure. Administration occurs after the procedure, for example, for at least 48 hours, at least 7 days, at least 14 days, or at least 28 days. Administration after the procedure may occur continuously or discretely, e.g., once, twice, three times, or four times daily or every other day. An exemplary dosing schedule after a procedure is administration every 6 hours for 2-30 days, preferably 2-7 days.

In another aspect, the invention features a method for treating a sickle cell anemia crisis in a patient having a sickle cell anemia disease. The method involves administering to the patient having a sickle cell anemia crisis or who is at risk for developing a sickle cell anemia crisis a therapeutically effective amount of a glycoprotein IIb/IIIa antagonist, e.g., xemilofiban.

The invention further features a method for treating heparin-induced thrombotic thrombocytopenia (HITT) in a patient. This method involves administering to the patient having HITT or who is at risk for developing HITT a therapeutically effective amount of a glycoprotein IIb/IIIa antagonist, e.g., xemilofiban.

In another aspect, the invention features a method for treating idiopathic thrombotic thrombocytopenia (ITTP) in a patient. The method involves administering to the patient having ITTP or who is at risk for developing ITTP a therapeutically effective amount of a glycoprotein IIb/IIIa antagonist, e.g., xemilofiban.

In another aspect, the invention features a method for treating tissue graft or organ transplant rejection in a patient, e.g., rejection of liver, kidney, heart, or lung transplants. The method involves administering to the patient having tissue graft or organ transplant rejection or who is at risk for developing tissue graft or organ transplant rejection a therapeutically effective amount of a glycoprotein IIb/IIIa antagonist, e.g., xemilofiban. The method may also include the administration of other anti-rejection drugs, such as Anti-CD40L, Anti-B7, Anti-TCR, a Calcineurin Inhibitor, tacrolimus, an Anti-cytokine/cytokine receptor, a steroid, Anti-CD154 monoclonal antibody (MAb), Anti-CD80 MAb, Anti-CD80/CD86 MAb, Anti-CD80/CD86 MAb, CTLA-4Ig, LEA-29Y, antisense ICAM-1, Anti-CD11a MAb, Anti-CD45RB MAb, Anti-CD45R MAb, LDP-01 Anti-integrin MAb, and methylprednisolone.

In yet another aspect, the invention features the use of a glycoprotein IIb/IIIa receptor antagonist in the preparation of a medicament for arterial vessel pacification or for the treatment of a sickle cell anemia crisis, heparin-induced thrombotic thrombocytopenia (HITT), idiopathic thrombotic thrombocytopenia (ITTP) or tissue graft or organ transplant rejection.

Exemplary glycoprotein IIb/IIIa antagonists for use in the methods and compositions of the invention include xemilofiban, abciximab, cromafiban, elarofiban, orbofiban, roxifiban, sibrafiban, RPR 109891, and tirofiban. Other glycoprotein antagonists are described herein. In various embodiments of the above aspects, the glycoprotein IIb/IIIa antagonist provides at least 80% inhibition of platelet aggregation in the patient. Exemplary routes of administration include oral, intravenous, local (e.g., in a stent), intra-coronary infusion, sublingual, or subcutaneous routes. The methods described herein may further include administering to the patient a therapeutically effective amount of an anticoagulant (e.g., aspirin, warfarin, a combination of aspirin and warfarin, or a non-fractionated or fractionated heparin, as described in WO 97/35592); a statin; a thienopyridine; a thrombin inhibitor (e.g., a recombinant hirudin); a factor Xa inhibitor; agonists of purinergic receptors, antagonists of CD40 or CD40 ligand (CD40L) or compounds that disrupt (e.g., reduce) the interaction of CD40 and CD40L, or an eicosanoid related target (e.g., COX inhibitors, PGE1 agonists, PG synthase inhibitors, TX synthase inhibitors, and TXA2 antagonists). Exemplary fractionated heparins are low molecular weight heparins, such as ardeparin, certoparin, dalteparin, enoxaparin, nadroparin, reviparin, or tinazaparin. Examples of thienopyridines include clopidogrel, ditazole, pirozadil, sarpogrelate, and ticlopidine. Exemplary thrombin inhibitors include argatroban, dermatan, desirudin, efegatran, inogatran, lepirudin, melagatran, mesoglycan, PEG-r-hirudin, and reviparin. Factor Xa inhibitors include, for example, danaparoid, fondaparinux, and tifacogin. Examples of purinergic receptor agonists include adenosine and adenosine analogs, e.g., 2-(N-pyrazolyl) derivatives of adenosine (e.g., CVT 3146), and 2-propynylcyclohexyl-5′-N-ethylcarboxamido derivatives of adenosine (e.g., ATL-146e (4-(3-[6-amino-9-(5-ethylcarbamoyl-3,4-dihydroxy-tetrahydro-furan-2-yl)-9H-purin-2-yl]-prop-2-ynyl)-cyclohexanecarboxylic acid methyl ester), and ATL-193). Examples of antagonists for CD40, sCD40L, and rsCD40L and compounds that disrupt the interaction include monoclonal antibodies (e.g., Antora and IDEC 131 and see US 2002/0031512 and WO01/34649), free CD40, and antisense nucleic acids. Exemplary eicosanoid related targets include alprostadil, beraprost, carbasalate, cloricromene, epoprost, etersalate, iloprost, indobufin, indometacin farnesil, limaprost, ozagrel, pamicogrel, picotamide, ramatroban, terbogrel, and triflusal. The compounds of the invention may be administered in combination (admixtures) from a single injection device or syringe. The therapeutic compounds of the invention may be administered as pharmaceutically acceptable salts or derivatives.

By “arterial vessel pacification” is meant the reduction or prevention of the interaction of activated platelets with leukocytes and/or the arterial vessel wall, for example, by inhibiting glycoprotein IIb/IIIa receptors and thereby reducing inflammatory cascades.

By “cardiac interventional procedure” is meant a medical or surgical procedure that repairs or prevents damage to the heart and associated arteries and veins.

By “pharmaceutically acceptable salt” is meant a non-toxic salt of a compound of the invention formed, e.g., from non-toxic inorganic or organic acids. Such non-toxic salts include, for example, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Other pharmaceutically acceptable salts are known to those skilled in the art.

By “sickle cell crisis” is meant an adverse condition resulting from sickle cell red blood cells (RBCs) typically characterized by intense pain. In a crisis, abnormal, elongated hemoglobin polymer structures distort the shape of RBCs. These abnormal RBCs can damage the vessels around them and the tissues that depend on the vessels for oxygen and nourishment.

By “therapeutically effective amount” is meant an amount of a pharmaceutical composition sufficient to produce a preventative, healing, curative, stabilizing, or ameliorative effect either in the treatment of a disorder or in the treatment of symptoms of a disorder.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of the disorder; and supportive treatment, that is, treatment employed to supplement another specific therapy directed toward the improvement of the disorder. The term “treatment” also includes symptomatic treatment, that is, treatment directed toward constitutional symptoms of the disorder.

By “unwanted platelet aggregation” is meant the aggregation of platelets, e.g., in blood vessels, in a patient that causes a detrimental effect to the patient.

As used herein the term “pharmaceutical composition” encompasses compounds of the invention together with pharmaceutically acceptable carriers and excipients.

Other features and advantages of the invention will be apparent from the following description and claims.

### BRIEF DESCRIPTION OF THE DRAWINGS

FIG. 1 is a schematic diagram of the induction of inflammation by the release of CD40L from activated platelets.

FIG. 2 shows graphs demonstrating that CD40L is predominantly found in platelets.

FIG. 3 is a schematic diagram of the functions of released sCD40L.

### DETAILED DESCRIPTION OF THE INVENTION

Certain medical and surgical procedures and medical conditions may cause undesirable platelet aggregation in individuals. Accordingly, the invention features a method for preventing platelet aggregation in patients at risk thereof by administering a therapeutically effective amount of a glycoprotein IIb/IIIa receptor antagonist, e.g., xemilofiban.

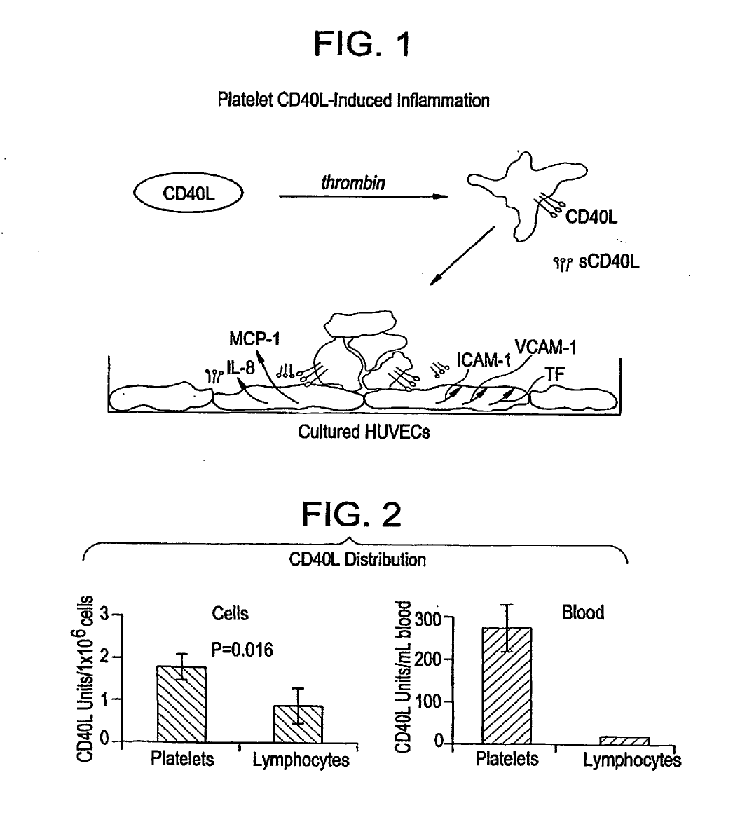

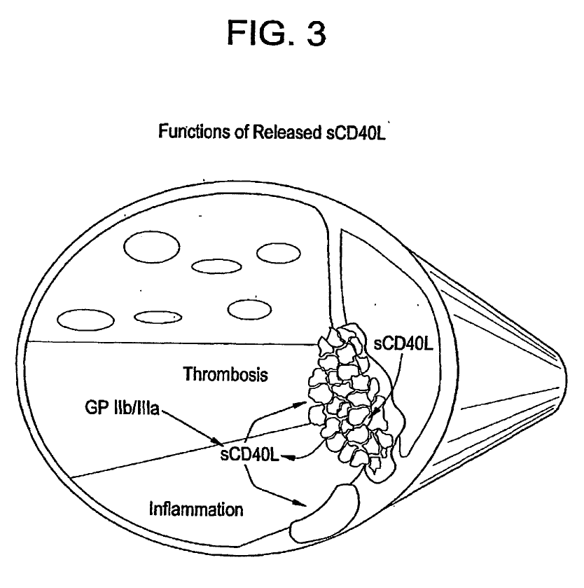

Platelet stimulation and subsequent aggregation are known to cause the expression or release of several factors that could affect vascular pathology. These include TXA2, a co-stimulator of platelets that has vasoconstrictive activity; P-selectin, an α granule protein that mediates platelet rolling, leukocyte adhesion, and coagulation; ADP and serotonin; which amplify platelet aggregation; platelet-derived growth factor, a growth factor for vascular cells; and CD40L, a member of the tumor necrosis factor family of proteins (reviewed in Gresele P, Page C, Fuster V. Platelets in Thrombotic and Non-thrombotic Disorders. New York, N.Y.: Cambridge University Press; 1992). Although any of these factors could contribute to long-term vascular pathologies, CD40L appears to be particularly relevant because this protein is now known to be prothrombotic (Andre P, Prasad K S, Denis C V, et al. CD40L stabilizes arterial thrombi by a beta3 integrin-dependent mechanism. Nat. Med. 2002; 8:247-252) and proinflammatory (Schonbeck U, Sukhova G K, Shimizu K, et al. Inhibition of CD40 signaling limits evolution of established atherosclerosis in mice. Proc Natl Acad Sci USA. 2000; 97:7458-7463), to have a proven role in atherosclerotic lesion progression (Schonbeck U, Varo N, Libby P, et al. Soluble CD40L and cardiovascular risk in women. Circulation. 2001; 104:2266-2268), and to be a risk factor for cardiovascular events (FIGS. 1-3). The majority of CD40L is found in platelets (FIG. 2) and thus released under conditions of activation and aggregation. Inhibition of the glycoprotein receptors reduces inflammatory cascades and thus passivates the vessel wall by interrupting the interactions of platelets with leukocytes and vessel walls.

### Treatments

Antagonists for the glycoprotein IIb/IIIa receptor are known in the art. Such antagonists include, without limitation, those disclosed in U.S. Pat. Nos. 5,470,849; 5,463,011; 5,455,243; 5,451,578; 5,446,056; 5,441,952; 5,422,249; 5,416,099; 5,405,854; 5,397,791; 5,393,760; 5,389,631; 5,380,713; 5,374,622; 5,358,956; 5,344,783; 5,340,798; 5,338,723; 5,334,596; 5,321,034; 5,318,899 (e.g. cyclic heptapeptides Mpr-(Acetimidyl-Lys)-Gly-Asp-Trp-Phe-Cys-NH₂, Mpr-(Acetimidyl-Lys)-Gly-Asp-Trp-Phe-Pen-NH₂, Mpr (Phenylimidyl-Lys)-Gly-Asp-Trp-Phe-Pen-NH₂, and Mpr-(Phenylimidyl-Lys)-Gly-Asp-Trp-Phe-Cys-NH₂, wherein Mpr is mercapto propionyl); U.S. Pat. Nos. 5,312,923; 5,294,616; 5,292,756; 5,281,585; 5,272,158; 5,264,420; 5,260,307; 5,239,113 (e.g. Ethyl 3S-[[4-[[4-(aminoiminomethyl)phenyl]amino]-1,4-dioxobutyl]amino]-4-pentynoate (xemilofiban)), U.S. Pat. Nos. 5,227,490; 5,206,373; 4,703,036 (e.g. N-Methyl-D-phenylalanyl-N-[(1S)-1-formyl-4-guanidinobutyl]-L-prolinamide); EP 505 868 (e.g. ((1-(2-((4-(aminoiminomethyl)benzoyl)amino)-3-(4-hydroxyphenyl)-1-oxopropyl)-4-piperidinyl)oxy)-(S)-acetic acid); WO 9311152 (e.g. N-(2-(2-(((3-((aminoiminomethyl)amino)propyl)amino)carbonyl)-1-piperidinyl)-1-(cyclohexylmethyl)-2-oxoethyl)-(R,S)glycine); EP 333 356; and WO 9422820. Other antagonists include abciximab, cromafiban, elarofiban, orbofiban, roxifiban, sibrafiban, and tirofiban.

The compounds of the invention can be administered by any standard means for administering therapeutic compounds, including, without limitation, oral, sublingual, transdermal, intravenous, parenteral, subcutaneous, intramuscular, intraperitoneal, intracoronary infusion, and administration into the cerebrospinal fluid. Administrations may be accomplished using standard means, such as auto-injection devices, constant infusion pumps, and minipumps. The compounds of the invention, such as any of those described herein, may also be impregnated or coated on a medical device, such as a stent, as disclosed in U.S. Pat. No. 5,609,629, hereby incorporated by reference.

Dosages and timing of administration can be determined using routine methods for such determination. In various embodiments, the glycoprotein IIb/IIIa receptor antagonist, e.g., xemilofiban, is administered to a patient at least 60 minutes, e.g., at least 90 minutes, at least 3 hours, at least 6 hours, at least 12 hours, at least 24 hours, at least 36 hours, or at least 48 hours, prior to the patient undergoing a medical or surgical procedure. Therapeutic compounds may be administered, e.g., once, twice, three times, four times, or more a day. The compounds may also be delivered continuously, e.g., through time-release formulations, over a period of time. The treatment may continue, e.g, for at least 60 minutes, 3 hours, 6 hours, 12 hours, 24, hours, 48 hours, 7 days, 14 days, or 28 days, after the procedure. For treatment after the procedure, the antagonist may be administered continuously, e.g.; through an intravenous infusion, or discretely, e.g., at least once daily (such as twice, three times, or four times daily) or every other day. For example, treatment after a procedure may include administration of the glycoprotein IIb/IIIa receptor antagonist, e.g., xemilofiban, every 6 hours for 2-30, preferably 2-7 days. For patients at high risk, e.g., diabetics, the treatment may be as long as 30 days. Typically, a 30-day treatment of high risk patients will also include a direct thrombin inhibitor or a Factor Xa inhibitor. The exact times of administration after the procedure may depend on the patient and the type of the procedure. The administration of antagonist administered after the procedure is designed to minimize prothrombotic events. The amount of antagonist administered is, e.g., 0.5 mg/dose, 1 mg/dose, 2.5 mg/dose, 5 mg/dose, 10 mg/dose, 20 mg/dose, 40 mg/dose, or even 80 mg/dose. Other dosages may be determined by one skilled in the art. The dosage regimen may be designed to prevent “troughs” or reduced periods of platelet inhibition that may be prothrombotic. In addition, it may be desirable to dose the patients in order to provide for rapid reversal of anti-thrombotic activity. Treatment may, for example, inhibit at least 60%, 70%, 80%, 90%, or 95% of platelet aggregation in a patient.

In one embodiment, the glycoprotein IIb/IIIa receptor antagonist is administered at least 60 minutes before a medical or surgical procedure. This dosage, called a loading dose, preferably inhibits 80% of platelet aggregation. A loading dose is required to achieve maximal plasma concentration in the shortest time frame thereby confering the greatest degree of protection to the patient by maximally occupying glycoprotein IIb/IIIa receptors present on activated platelets. This protection leads to “passivation” of the platelets and the blood vessel wall thus reducing or preventing the local and systemic releases of CD40/sCD40L, white blood cell adhesion molecules (e.g., ICAM-1, VCAM-1, IL-8, TF, and MCP-1), white blood cell and platelet coaggregates, and P-selectin expression. Without a loading dose, steady state plasma concentrations would not be achieved until 4-5 plasma half-lives or about 24-30 hours. In a preferred embodiment, xemilofiban is the glycoprotein antagonist employed since it also inhibits P-selectin and the alphaVbeta3 receptor, which are also involved in platelet interactions.

The glycoprotein IIb/IIIa receptor antagonist may be co-administered with other therapeutic compounds. In addition, therapeutic compounds may be administered in pharmaceutically acceptable carriers, such as sterile water or isotonic saline. Additional compounds that may be administered with the glycoprotein IIb/IIIa receptor antagonist include, without limitation, heparins (fractionated, e.g., low molecular weight heparins, or unfractionated), anticoagulants (e.g., aspirin), statins (e.g., atorvastatin, fluvastatin, lovastatin, pravastatin, or simvastatin), thienopyridines, thrombin inhibitors, factor Xa inhibitors, and eicosanoid related targets. Exemplary low molecular weight heparins include ardeparin, certoparin, dalteparin, enoxaparin, nadroparin, reviparin, or tinazaparin. Examples of thienopyridines include clopidogrel, ditazole, pirozadil, sarpogrelate, and ticlopidine.

The glycoprotein IIb/IIIa receptor antagonist may also be administered in combination with a purinergic receptor agonist. Desirable receptor targets include the A_{2A} and A₃ receptors. Exemplary purinergic receptor agonists include adenosine (e.g., in intravenous form) and adenosine analogs (e.g., CVT 3146, ATL-146e, and ATL-193).

Alternatively, the glycoprotein IIb/IIIa receptor antagonist may be administered in combination with a compound that antagonizes CD40 or CD40L or disrupts the CD40-CD40L interaction, e.g., antibodies (such as monoclonal antibodies) for CD40 or CD40L, CD40 antagonists, free receptor, or other molecules that bind to, degrade, or prevent the transcription or translation of CD40 or CD40L (e.g., antisense nucleic acids).

### Medical or Surgical Procedures

Medical or surgical procedures that may cause unwanted platelet aggregation include, for example, cardiac interventional procedures, tissue or organ transplantation and angioplastic procedures. These procedures include, without limitation, percutaneous transluminal coronary angioplasty with or without placement of an intracoronary stent, cardiac bypass surgery, hemodialysis, extra-corporeal circulation associated with a surgical procedure, intracranial angioplasty, and angioplasty on peripheral arteries.

### Medical Conditions

Medical conditions that are preventable or treatable according to the invention include, without limitation, sickle cell anemia crisis, heparin-induced thrombotic thrombocytopenia (HITT), idiopathic thrombotic thrombocytopenia (ITTP), stroke, atherosclerosis, angiogenesis, thrombosis, thromboembolic conditions such as deep venous thrombosis, pulmonary embolism or thrombophlebitis, disseminated intravascular coagulation or thromboembolic syndromes associated with cancer, sepsis, or obstetrical complications, peripheral arterial occlusive disease, acute coronary syndromes such as unstable angina and myocardial infarction, diabetes, or tissue damage caused by phospholipases A₂ (PLA₂).

Regarding sickle cell anemia, damaged RBCs formed during a sickle cell crisis can cause thrombosis (clotting) and then secondary ischemic damage to the adjacent and surrounding tissues, causing infarction (cellular death). Abnormal interaction of sickle red blood cells (SS RBC) with the vascular endothelium has been implicated as a factor in the initiation of vasoocclusion in sickle cell anemia. Both von Willebrand factor (vWf) and thrombospondin (TSP) play important roles in mediating SS RBC-endothelium interaction. SS RBCs can bind to the endothelium via alphaVbeta3 receptors. Xemilofiban blocks both glycoprotein IIB/IIA and alphaVbeta3 receptors. Blockade of glycoprotein IIb/IIIa receptors therefore constitutes a potential therapeutic approach to prevent SS RBC-endothelium interactions under flow conditions.

### Tissue'Graft and Organ Transplant Rejection

Tissue graft and organ transplant rejection involves a strong inflammatory process, in which glycoprotein IIb/IIIa receptor antagonists may have therapeutic efficacy through their activity on platelets, which, in part, prevents the release of sCD40L. Glycoprotein IIb/IIIa receptor antagonists may be administered in combination with other anti-rejection drugs including Anti-CD40L, Anti-B7, Anti-TCR (CD4, CD3), Calcineurin Inhibitors (such as Rapamycin (siromilus), tacrolimus (e.g., found on coated stents), Anti-cytokines/cytokine receptors, steroids (e.g., metoprolol), Anti-CD154 MAb, Anti-CD80 MAbs, Anti-CD80/CD86 MAbs, Anti-CD80/CD86 MAbs, CTLA-4Ig, LEA-29Y (mutant CTLA-4Ig), ISIS-2302 (antisense ICAM-1), Hu1124/Anti-CD11a MAb, Anti-CD45RB MAbs, Anti-CD45R MAbs, and LDP-01 Anti-integrin MAb.

All agents would be administered either before, during, or after the graft or transplant procedure, for example, tissue graft, organ transplant (e.g., kidney, heart, liver, or lung).

All agents will typically be given via oral, intravenous, or subcutaneous routes. In one example, patients unable to take oral tacrolimus capsules may be initiated with tacrolimus injection. In this example, the first dose is administered at least 6 hours after transplantation. The recommended starting dose is 0.03-0.05 mg/kg/day as a continuous intravenous infusion. Adult patients typically receive doses at the lower end of the dosing range. Continuous intravenous infusion should be continued only until the patient can tolerate oral administration of tacrolimus capsules. Concomitant adrenal corticosteroid Methylprednisolone therapy is recommended early post-transplant. A glycoprotein IIb/IIIa receptor antagonist, e.g., xemilofiban, will be given orally or intravenously 6 hours after transplantation and continued for 2-30 days.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are within the claims.

What is claimed is:

1. A method for inhibiting platelet aggregation in a patient undergoing a medical or surgical procedure, comprising administering to the patient, at least 60 minutes prior to the procedure, a therapeutically effective amount of a glycoprotein IIb/IIIa receptor antagonist.

2. The method of claim 1, wherein said glycoprotein IIb/IIIa antagonist provides at least 80% inhibition of platelet aggregation in the patient.

3. The method of claim 1, wherein said procedure is a cardiac interventional procedure.

4. The method of claim 3, wherein said cardiac interventional procedure is percutaneous transluminal coronary angioplasty.

5. The method of claim 3, wherein said cardiac interventional procedure is cardiac bypass surgery.

6. The method of claim 1, wherein said procedure is an angioplastic procedure.

7. The method of claim 1, wherein said glycoprotein IIb/IIIa receptor antagonist is xemilofiban.

8. The method of claim 1, further comprising administering to said patient, prior to the procedure, a therapeutically effective amount of a thienopyridine.

9. The method of claim 8, wherein said thienopyridine is selected from the group consisting of clopidogrel, ditazole, pirozadil, sarpogrelate, and ticlopidine.

10. The method of claim 1, further comprising administering to said patient, prior to the procedure, a therapeutically effective amount of an anticoagulant.

11. The method of claim 10, wherein said anticoagulant is aspirin.

12. The method of claim 10, wherein said anticoagulant is a non-fractionated or fractionated heparin.

13. The method of claim 12, wherein said fractionated heparin is a low molecular weight heparin.

14. The method of claim 13, wherein said low molecular weight heparin is ardeparin, certoparin, dalteparin, enoxaparin, nadroparin, reviparin, or tinazaparin.

15. The method of claim 1, further comprising administering to said patient, prior to the procedure, a therapeutically effective amount of a purinergic receptor agonist.

16. The method of claim 15, wherein said purinergic receptor is an A_{2A} or A₃ receptor.

17. The method of claim 15, wherein said agonist is adenosine, CVT 3146, ATL-146e, or ATL-193.

18. The method of claim 1, furthering comprising administering to said patient, prior to the procedure, a therapeutically effective amount of a compounds that antagonizes CD40 or CD40L or that disrupts the interaction of CD40 and CD40L.

19. The method of claim 18, wherein said compounds is a monoclonal antibody, free CD40, or an antisense nucleic acid.

20. The method of claim 1, further comprising administering to said patient, prior to the procedure, a therapeutically effective amount of a thrombin inhibitor, a statin, a factor Xa inhibitor, or an eicosanoid related target.

21-70. (canceled)
